(19) United States
(12) Patent Application Publication
(10) Pub. No.: US 2010/0048873 A1
(43) Pub. Date: Feb. 25, 2010

(54) METHOD OF OBTAINING PLANT PROTEIN FRACTIONS WITH A MEDIUM MOLECULAR WEIGHT, PLANT PROTEIN FRACTION, AND ITS USE

(75) Inventors: Martin Lotz, Emlichheim (DE); Gerold Eggengoor, Wilsum (DE)

Correspondence Address: ROBERTS MLOTKOWSKI SAFRAN & COLE, P.C.;Intellectual Property Department, P.O. Box 10064, MCLEAN, VA, 22102-8064, US

(73) Assignee: EMSLAND-STAERKE GMBH, Emlichheim (DE)

(21) Appl. No.: 12/447,358

(22) PCT Filed: Sep. 24, 2007
(86) PCT No.: PCT/DE2007/001723

§ 371 Date: Apr. 27, 2009

(30) Foreign Application Priority Data
Oct. 26, 2006 (DE) 10 2006 050 620.0

(51) Int. Cl. C07K 14/415; C07K 1/30

(52) U.S. Cl. 530/370; 530/419

(57) ABSTRACT

A method of preparing a coagulated plant protein fraction with a medium molecular weight of between 14 to 97, by providing fruit juice in aqueous solution; precipitating a high-molecular-weight plant protein fraction whose bulk has a molecular weight of from above 100 to 600, by adjusting an acidic pH and/or a temperature above room temperature, and mechanically separating the fraction precipitated thus; precipitating a medium-molecular-weight coagulated plant protein fraction under warm conditions by treating, at pH 2 to 7, and between 60 to 90° C., the solution obtained after separation of the high-molecular-weight plant protein fraction and mechanically separating the medium-molecular-weight coagulated plant protein fraction with a molecular weight of between approximately 14 to 97, with the bulk of the molecular weight distribution being between 20 to 60. The plant protein fraction is usable as foodstuff, food additive, additive in pharmaceuticals, animal feed, in cosmetics, as industrial protein, as adhesive.

### BACKGROUND OF THE INVENTION

1. Field of Invention

The invention relates to a method of obtaining a tuber protein fraction with a high molecular weight of between 14 and 97, a tuber protein fraction obtained by such a method, and the use of such a tuber protein.

2. Description of Related Art

Proteins are vital chemical compounds for the entire living world, mostly with biochemical functions as enzymes, but also as storage substance (storage proteins), so to say, as resource reservoir for vitally important processes, primarily for growth and/or reproduction processes. Proteins are characterized by their molecular size, their composition as well as their secondary and tertiary structure. We talk of proteins if the chemical structure consists exclusively of amino acids and the chain length amounts to approximately 30 or more amino acids. Shorter chains are usually called peptides. However, there is actually no exact definition of this classification. It is rather arbitrary and only useful for some individual situations. Proteins are generated by mono-cellular creatures, e.g., bacteria and yeasts, plants but also by animals. They are indispensable to human and animal nutrition as well as health. Besides their chemical, nutritive and biochemical characteristics, proteins possess also so-called functional characteristics. Features like their water absorption capability, digestibility, water solubility, the creation and stabilization of foam as well as their emulsifying capabilities are characterized by the tertiary and secondary structure and make them applicable for technical applications. Thus, proteins are used in many technical fields, for instance as glue, emulsifying and thickening agents. If exposed to heat or acids or leaches their tertiary structure is severely and often irreversibly damaged. The secondary structure is destroyed by proteolysis occurring due to enzymes or highly alkaline or acid mediums. Thus, it is very important to maintain the proteins' functional characteristics during their isolation.

The state of the art is to isolate both animal and plant proteins. They are already used in manifold application fields, e.g., as foodstuff (tofu), animal feed, in cosmetics and as industrial protein (protein glue or similar). The protein's significance to diets, nutrition technology, e.g., as foaming agent, emulsifying agent, structural and/or texturing agent (e.g., gelatin for gummy bears and glaze), animal feed, cosmetics and medical products is unique and cannot be substituted by any other substance classes. The easiest way to obtain such functional proteins which posses good water solubility and emulsifying capabilities is to use milk or eggs and processes (no heavy changes in pH values, no high temperatures) that do not have any harmful effects to the product. In technical sectors caseinate is frequently used as glue for bottle labels; collagen is often used in cosmetics products.

It's a problem that especially animal proteins often trigger allergic reactions.

Cow milk proteins which are used quite frequently are feared by many consumers suffering from lactose intolerance or who are allergic to cow milk proteins. Additionally, animal proteins have the disadvantage carrying possible diseases (such as BSE, HIV or avian flu) or that they could be pathogen. Another disadvantage of animal proteins is that, due to ethical reasons, they are often not accepted by many population groups. For instance, skin creams which are based on collagen are proscribed in Asian and Muslim cultures for exactly these grounds. However, even in our cultures they cause various allergies. Furthermore, animal proteins are normally more expensive than plant proteins because plant proteins are by far more sustainable than animal proteins—plant proteins are cheaper to produce and are ideal for vegetarian or any other diets such as purine-reduced diets or nutrition. Thus, it makes sense to examine pant proteins in more detail.

Plant proteins, in particular high-quality potato proteins, avoid many of the above mentioned disadvantages of animal proteins. Many of them are hardly or not allergenic, i.e., they are not registered in the allergen list of the EU, are accepted in all cultures, and, due to the culture of particular tubers, such as potatoes, it is possible to guarantee organic products and products free of GM technology (non-GMO certificates).

From all industrially used animal proteins such as milk and whey products, gelatin, chicken egg protein, collagen etc. primarily milk proteins, mainly casein and its salts, chicken egg protein as whole protein or albumen as well as egg yolk and proteins isolated from butchery residuals such as gelatin, bone glue and collagen are used in technology sectors—i.e., in nutrition technology, technical sectors or similar. Today, isolated plant proteins used on a daily base only come from a limited number of plants.

Common isolated plant proteins stem from legumes, such as soy, sometimes also from peas and wheat. Isolates applied on a bigger scale are soy and wheat proteins, the so-called gluten. Gluten is a problematic protein because many people are allergic to gluten (celiac disease) and there is enormous demand for gluten-free plant protein. Soy protein, widely used as substitution product for animal protein as well as animal feed, is not indisputable because it contains hormone-active substances.

Further isolated plant proteins of subordinate significance stem from rape, lupines and other legumes or potatoes. The quality of many commercially available plant proteins, particularly potato proteins, is not yet satisfactory, although this is desirable. The reasons for that are various.

### SUMMARY OF THE INVENTION

In the following, the invention is described in more detail on the basis of potatoes—however, it is in no case limited to this species alone, but can, as it is apparent to the expert, easily be adjusted to other tubers and plants.

For producing plant proteins from fruit juice which is generated by educing, drawing out or squeezing relevant plant sections, especially fruits, two different technical methods are used.

1. Heat coagulation of the proteins in the fruit juice by inactivation of the enzymes

or

2. Precipitation of the proteins from the fruit juice with acidic pH.

In connection with the invention, tuber parts refer to the storage organs of tubers (e.g., potatoes, tapioca, girasol, yams, sweet potato, taro, bitter manioc, yams, papyrus, ullucus tuberosus c.-bulbous nasturtium, oxalis tuberosa m., etc.).

In connection with the invention, fruit juice refers to both sap squeezed from the plant sections and plant protein solutions which are educed (drawn out) from plant sections by the use of aqueous mediums. Plant sections need to be educed whenever the liquid content of the relevant plant section is inappropriate or residual protein needs to be mobilized.

Up to now, such plant proteins have usually been generated by heat/acidic treatment of the fruit juice and by separating the precipitated protein directly from the fruit juice.

The result of the thermal precipitation by use of which proteins are educed from the fruit juice is a slightly soluble product with insufficient functionalities that is indigestive, has a heavy flavor, and contains harmful substances.

With higher temperatures the protein will excessively be damaged and its precious characteristics which make it so useful for food industries increasingly destroyed: neutral taste, bright color, solubility, all other functionalities too, the structure becomes horny and digestibility decreases.

Typical negative substances coming into being as concomitant substances in precipitated plant proteins are for instance: trypsin inhibitors, proteins inhibiting the proteopeptic enzyme trypsin and, thus, also digestion. Trypsin inhibitors can only be rendered harmless by targeted deactivation processes, e.g., by heating treatment with 70° C. or higher.

Additionally, the following substances may occur: Tannins/tannic acids inhibiting digestion as well as the ingestion of iron and inactivating digestive enzymes; toxic glycoalkaloids, such as solanine in solanaceous herbs; protease inhibitors and polyphenols.

Additionally, many plant proteins generated by thermal precipitation of protein solutions in alkaline milieus (also including the pasteurization process sometimes necessary) possess higher contents of lysine alanine, an anti-nutritive condensate whose content should be as low as possible.

Another problem of the plant proteins currently available is their strong inherent taste such as soy protein. Thus, many applications are not possible or the quantities used are restricted, which is also often disadvantageous.

Concerning food applications it must be noted that the most plant proteins are not wholesome, i.e., they do not contain all amino acids, in particular the 8 so-called essential amino acids the human body cannot produce itself and which must thus be supplied externally. The quality of plant proteins is generally lower than those of animal proteins—the best quality rating of all plant proteins have potato proteins.

Today, there are already some types of potato protein which are produced by strong coagulation with high temperatures and dehydration. These proteins are approved according to the Novel Food-VO. Hydrolyzates from this potato protein are also approved according to this Novel Food-VO. Disadvantages of this well-known protein are its relatively dark color, its taste for potatoes and the hot “ticklish” taste. This is caused by heavily denaturated proteins, modifications of the tertiary structure and the loss of technological functionalities, e.g., bonding capacities of the named potato protein concerning water and/or oils have diminished.

Therefore, many attempts to regain solubility and, thus, relevant technological functionalities aimed at hydrolyzing this potato protein. However, this causes certain problems which are typical for all types of protein hydrolyzates, namely allergenicity as well as the bitter taste due to peptides and, certainly, high efforts due to multiple treatments which cause very high manufacturing costs and, thus, also high market prices.

One additional disadvantage of the methods used so far for isolating potato proteins is that glycoalkaloids which are toxic to human beings as well as animals (of which solanine is the most popular) must be separated in extra process steps. This is done by selected elutriation processes with much washing water in acidic milieus with only little quantities of dry substance because glycoalkaloids are hardly soluble. Another possibility is to use expensive solvents which need to be regained and reprocessed. Both procedures are very time and cost consuming.

The brown color of known potato proteins, which is caused by polyphenols oxidized into poly-quinones and melamines, is another decisive disadvantage which has prevented the principally high quality potato protein from being used in the food industry. Additionally, polyphenols are also responsible for the bitter taste.

These known potato proteins produced by thermal precipitation additionally possess only little functionalities and are, due to their high degree of denaturation, hard to digest, i.e., they are hardly or not suitable as milk protein substitute.

The same applies to proteins of other plants, such as wheat, rape, soy etc. which also possess harmful concomitant substances. It is the objective of the invention to provide plant proteins which possess better functionalities and which avoid the disadvantages of the plant proteins already known by use of processes that are less expensive and complex.

According to the invention the protein is selectively fractionated by protein-friendly, surprisingly simple methods, which lead to a protein fraction that is suitable for use in foods and has adequate functionalities. It is important that, as separation technique for the different proteins, fractionation is used. Fractionation by selective adjustments of pH values and temperatures is an efficient and cheap procedure that allows for simple and surprisingly selective protein fractionation on large technical scales.

The fraction according to the invention can also be obtained by gradual membrane filtration or gradual precipitation with solvents. Both methods will consume more time and efforts. It is often advantageous to separate mechanically with decanters which are able to separate large amounts of material into solids and overflow (both continuously and fast).

With many tuber sections—such as potatoes—it makes sense to use an “anoxic process control,” at least until the polyphenols (PP) are separated from the desired protein fraction (actually from the tuber fruit juice) and/or the proteins which catalyze the enzymatic oxidation are coagulated, and thus, there are no more oxidizing processes both enzymatic catalyzes and/or non-enzymatic catalyzes possible. The anoxic process is carried out by excluding aerial oxygen e.g., by adding nitrogen, flue gas among other gases, thus, squeezed out the air of material, pipes and apparatuses, additionally by use of airproof apparatuses that only allow the natural oxygen content of the tuber sections in the process, as well as by intercepting the oxygen contained in the process by use of auxiliary chemicals such as antioxidants (ascorbic acid, citric acid, SO2, sodium bisulfite amongst others) and/or antifoam agents.

It is certainly possible to apply all three principles mentioned in any desirable order. The plant protein fraction according to the invention is particularly suitable for food, food additives, pharmaceutical additives, animal feed, cosmetics products, as technical protein, and glue because, on the one hand, it is available in adequate quantities, and on the other hand, provides sufficient functionalities.

The selective fractionation of tuber protein, such as in native potato salad/fruit juice of potatoes is carried out with two underlying directives: On the one hand, undesired and distracting fractions need to be (previously) separated from the fruit juice and, on the other hand, to isolate the desired, i.e., only and exactly this, fraction from the fruit juice. The method according to the invention is as follows: from one process step to another precipitation conditions are increased in order to isolate fractions with exactly those proteins that have the next lower molecular weight.

According to the invention, the method comprises:
Grinding the entire or peeled or otherwise apportioned fruit in order to release the fruit juice enclosed in the cells, if necessary by adding (watery) solvent;
Completely or partially separating the now available fruit juice and/or the protein solution containing the entire protein,
Mechanical separation of a first insoluble fraction of the protein after the first precipitation. This can directly be done with the fruit juice, after adjustment of a particular pH value, a certain temperature, which is only slightly above the room temperature, or a combination of both. For instance, with potatoes, the glycoalkaloids absorb at the separated protein, enrich themselves in this separated fraction and degrade in the liquid phase, whereby proteins with lower molecular weight remain in the supernatant. Thus, expensive processes to separate undesired harmful substances are avoided due to the absorption in this fraction.
Mechanically separating the protein's target fraction from the supernatant resulting from the first precipitation. The precipitation of the target fraction can be achieved by adjusting an appropriate pH value and/or an, in relation to the first precipitation, increased temperature.
If necessary, washing and drying the target protein fraction.

### Step 1

The first step is used to separate large proteins. This fraction also contains the so-called polyphenols whose enzymatic and subsequent non-enzymatic oxidation causes the brown color and the bitter taste. With potatoes this fraction is a type of sludge which comprises glycoalkaloids, has a dark undesired color, potato taste, polyphenols, protein complexes and is only suitable for animal feed.

The pure protein content (N*6.25) is low (approximately 45%).

This can be achieved by:
Centrifuging the pure fruit juice. Here, the lowest amount of protein is separated, but at least in “mono-fractions”
   or
Adjusting the acid pH value between pH 2 to 7, preferably between 4-6 and acidic precipitation of the large proteins, whereby the precipitated material is then mechanically separated, for instance by centrifugation. It is important that there will be not too much (which additionally reduces the already very low rate of yield) and not too little (which deteriorates the pureness of the protein of the desired fraction as well as other quality parameters) precipitation material
   or
By slightly increasing, in relation to the room temperature, temperature by 25 to 50° C.
By an appropriate combination of an increased temperature (25-50° C.) and an acidic pH value of 2 to 7.

One particular advantage of this process step is its extraordinary simplicity, i.e., in terms of machines, materials and energy consumption.

The safest and cheapest way of avoiding this undesired protein fraction in the desired fraction is to separate it previously from the fruit juice under defined process conditions, namely adjusting and monitoring both pH value and temperature. This first step is used for separating polyphenols and glycoalkaloids as well as low-grade protein fractions, whereby an inevitable loss of proteins has to be accepted here.

### Step 2

Here, the target protein fraction is isolated by adjusting a pH value which is suitable for precipitation together with a thermal precipitation of the supernatant of step 1. For that, a pH value is selected around the isoelectric point of the protein, in combination with a temperature increase above room temperature.

Accordingly, this pH value complies with the precipitation conditions from the acidic under increased temperatures—accessible at a pH value between 2-6 and 50 to 85° C. In terms of the product characteristics, it should additionally be mentioned:

Surprisingly, with the method according to the invention which does not require any expensive or complicated processes or process steps and which is very fast, it is possible to achieve the desired target with a combination of simple process steps. Beyond that, the required apparatuses are cheap and operating expenses are low.

It is very advantageous that no chemical additives have to be applied in the process, such as enzymes, disinfectants, brighteners.

Concerning the product's characteristics and/or features, it is also surprising that, by means of that fractionation, not only proteins with a special molecular weight range are isolated, but also a fraction that possesses all required characteristics: highly nutritive, neutral color, no distinct plant and/or potato taste, low lysine-alanine contents, little glycoalkaloids as well as technological functionalities such as water retention and oil-binding capabilities (emulsifier). This is particularly surprising because the precipitated protein fraction is nearly water-insoluble.

In the following the invention is now described in more detail by means of some examples (to which the invention is by no means restricted to).

### DETAILED DESCRIPTION OF THE INVENTION

The process conditions for the first step, the first fractionation step, are: Room temperature up to 50° C., pH 2-7, and separator as centrifuge. This so separated protein fraction with an MG of approximately 100-600 kD can, for instance, be used as common animal feed (currently, most of the unseparated potato protein is used as animal feed). Under these conditions, proteins such as glycol-proteins, phosphor-proteins, lipoproteins, metal-coordinated proteins etc., that are connected to none protein-type molecules are separated together with the high-molecular potato protein. Here, also the largest part of the glycoalkaloids and polyphenols is separated.

In the second step, the target fraction is separated by: precipitating with pH values of 2-6, preferably pH 3.5 to 5.5 and 50-85° C., preferably 75 to 85° C., separating the precipitated potato protein fraction from the potato juice using a decanter centrifuge. One of the peculiarities of the potato protein is that, the more acidic the adjusted pH value is, the more protein is precipitated. With all other products, both of plant and animal origin, at the isoelectric point, precipitation is at its maximum and also in ideal terms of the “flocculent characteristics.”

With potato protein contained in potato salad, this is pH 5.4, nearly the native pH value of potato salad (pH 5.6).

Potato protein also has another peculiarity. Thermal coagulation, above 40° C., is irreversible with potato protein. The parameters also guarantee the product's microbiological pureness, i.e., it is not necessary to additionally pasteurize the product. High or very high temperatures as well as acidic milieus are avoided, thus only little lysino-alanine will be produced.

The protein product generated had a protein content of approximately 75% as dry substance.

This pure protein fraction with an MG of 14 to 97 is cleaned/washed with mains water to reach isolate quality, i.e., >80, preferably >85% protein in the dry substance, both cold (room temperature) or hot (preferably 60° C. to 85° C.), pH neutral or acidulated to pH 4 to 7, i.e., at conditions near precipitation conditions but somewhat milder. Typically, this process is carried out in several steps, for instance with decanters which are connected in series, whereby one half of the washing water is supplied in front of the decanter or in the reverse flow, i.e., the entire washing water is supplied in front of the second decanter and discharged from the process in the upper flow section of the first decanter.

The potato protein fraction manufactured according to the invention avoids all disadvantages of the conventional potato proteins mentioned above (color, bitterness, allergenicity, glycoalkaloids, anti-nutritive decomposition products, inherent taste, and loss of functionalities) by elutriating or separating these substances together with the proteins of the first step. Some of the enzymes could be anti-nutritive; here the protease inhibitors are again outstanding, which have antibacterial functions in the potato tuber. According to references in technical publications, these enzymes have a low MG assuring that they are not contained in the fraction described here. Beyond that, the heat treatment causes denaturation and inactivation, which can also be deduced from the protein fraction's low solubility of 2 to 5%.

Harmful substances: Besides normal environmental toxins, such as heavy metals and pesticides potatoes, harmful substances, such as glycoalkaloids (solanine etc.) are present. As described above, these substances are separated in step 1. Allergic potentials of such potato proteins are not known. Thus, this potato protein fraction is suitable for producing special allergen-free, vegetable food and cosmetic products.

According to SDS-PAGE the molecular weights of the various proteins of the potato protein fraction according to the invention amount to 14, 20, 22, 40, 97 kD. Basically, the fraction consists of patatin (40 kD) and 20/22 kD, the rest is generally negligible.

Potato protein fractions manufactured in this way had the following characteristics:

Max. 150 ppm glycoalkaloids
Max. 1% starch
Max. 1% sugar
Max. 1% crude fiber*
Isoelectric point: approximately 5.4
pH value of 4.0 to 6.0

### Max. 5% ash:

Emulsifying capacity: 1:4:4 to 1:4:6
Solubility—2% to 5% (in water with room temperature and also in hot water)
Water-binding capacity: 1:4 to 1:5

### Measurement of Emulsifying Capacity:

25 g of protein are suspended in 100 g water using an Ultra-turrax. Subsequently, oil is added slowly and in portions while continuing dispersing (e.g., sunflower seed oil, rape oil, olive oil etc.) until the emulsion breaks.

The oil-binding and emulsifying capacity is indicated with regard to the concentration of the 3 substances with the maximum oil-binding capacity. 1:4:6 means that a mixture of 1 part protein and 4 parts water can bind 6 parts of oil, i.e., after adding more than 150 g oil to the above mentioned suspension the test emulsion will break.

### Determination of Water-Binding Capacity:

5 g of protein are weighted into 95 g water and the suspension is stirred for 1 h. Then it will be centrifuged (20 min., 3,500 g), the supernatant will carefully be decanted (if necessary the remaining liquid has to be removed by pipettes) and the wet protein will be weighted.

(Wet weight−dry weight)/dry weight=water-binding capacity

Amino acid composition of the obtained potato protein fraction with a molecular weight between 14 and 97 kD was as follows (fluctuations typical for natural products) with essential amino acids underlined:

| | |
|---|---|
| Ala | 3.7-3.9 |
| Arg | 3.9 to 4.4 |
| Asp | 9.8 to 12.6 |
| Cys | 1.4 to 1.7 |
| Glu | 8.9 to 9.5 |
| Gly | 4.7 to 5.0 |
| His | 1.9 to 2.3 |
| Ile | 4.9 to 5.4 |
| Leu | 8.6 to 9.6 |
| Met | 1.7 to 1.9 |
| Phe | 5.8 to 6.0 |
| Pro | 4.1 to 4.5 |
| Ser | 4.3 to 4.8 |
| Thr | 4.6 to 5.2 |
| Try | 1.0 to 1.2 |
| Tyr | 4.6 to 5.0 |
| Val | 5.9 to 6.9 |
| Lys | 7.3 to 7.4 |

The entire content of essential amino acids amounts to 40.8% to 43.1%. The sum of the amino acids in the dry substance is 95.6%, in OS 91.5%, raw protein (N*6.25) 85.4% in the dry substance.

In this potato protein fraction, the high nutritional value of the potato protein is made accessible to the people and, simultaneously, a protein which offers the mentioned technological advantages is made available to fabricators who produce the converted food products.

With increased temperatures, the protein is excessively damaged and its precious characteristics which make it so useful for the food industries are increasingly destroyed: neutral taste, bright color, solubility, all other functionalities too, the structure becomes horny and digestibility decreases. The protein fraction, which remains soluble even under these conditions, is left over in the fruit juice of the potato.

### Production of Potato Protein

50 kg potatoes of the type Saturna were washed and crushed in a mill; the fruit juice of the potato obtained by pressing the ground parts in a centrifuge by adding sodium bisulfite. 25 l of this fruit juice of the potato were adjusted to a pH value of 4.6 with salt acid and the resulting precipitate again been centrifuged with protein sludge, polyphenols and glycoalkaloids. The supernatant was heated for 30 minutes to 75° C. The precipitate then washed in two additional washing steps at a temperature of 70° C. and the result was 0.25 kg dry substance in the form of bright powder.

### Using the Potato Protein Fraction as Emulsifying Agent in a Hypo-Allergenic Salad Dressing

43.2% rapeseed oil was stirred with 10% salt egg yolk, 34.08% water, 6.00% potato protein, 1.15% NaCl, 7.2% sugar, 0.5% potato fiber, 0.03% paprika, 0.01% carotene, 0.05% white pepper, 7.14% 10% spirit vinegar and 0.64% hot mustard. The result was a hypo-allergenic salad dressing in which it was possible to avoid starch and proteins both of which are usually used as thickening and emulsifying agents. The dressing's emulsifying stability and storability was good and its taste did not cause any complaints.

### Using the Potato Protein Fraction as Emulsifying Agent in a Hypo-Allergenic Tomato Ketchup

30% double concentrated tomato puree, 35.4% water, 9.5% 10% spirit vinegar, 19.00% sugar, 2.3% salt, 2.5% potato protein, 0.5% potato fiber and 0.8% citric acid were mixed. Here, the result was ketchup free of preservatives which was obtained by replacing the commonly used wheat starch (for gluten-allergic persons).

### Carbohydrate-Reduced Noodle Dough

150 g whole egg, 400 g potato protein, 5 g guar flour, 100 g water, 60 g potato fibers and 6 g salt were kneaded; the result was noodle dough. This noodle dough was then formed into noodles and dried. The result was a product with low carbohydrate contents, particularly with a small percentage of quickly absorbable carbohydrates; suitable for weight reduction of diabetics.

### Protein-Enriched Cream-of-Carrots Soup

300 g carrots, 200 g potatoes, 40 g spring onions, 15 g parsley, 500 g water, 100 g potato protein, 10 g lime juice, 250 g milk, 100 g sour cream, 2 g black pepper and 17 g salt were mixed resulting in a soup of 1534 g. This soup is ideal for protein-enriched build-up diets.

Although the invention has been described by means of selective examples, experts will certainly recognize that manifold variations of the invention are possible. All of these variations are intended to be within the scope of the appended claims.

What is claimed is:

1. Method of obtaining a tuber protein fraction with a high molecular weight of between 14 and 97, comprising:
providing a tuber fruit juice in aqueous solution;
precipitating a high-molecular-weight tuber plant protein fraction whose bulk has a molecular weight above 100 to 600, by at least one of adjusting an acidic pH to 2-7 and/or a temperature to 25-50° C. and mechanically separating the fraction precipitated thereby;
precipitating a medium-molecular-weight coagulated tuber plant protein fraction under warm conditions by treating to a pH of 2 to 7, and to a temperature between 60 to 90° C., a solution being obtained after separation of a high-molecular-weight tuber plant protein fraction and
mechanically separating the medium-molecular-weight coagulated tuber plant protein fraction with a molecular weight of between approximately 14 and 97 kD, with the bulk of the molecular weight distribution being between 20 and 60 kD.

2. Method according to claim 1, comprising the further steps of washing the resulting medium-molecular-weight coagulated tuber plant protein fraction by at least one step involving slurrying with water at a neutral or acidic pH above the precipitation pH value at one of room temperature and a temperature below the precipitation temperature, and mechanically separating the medium-molecular-weight coagulated tuber plant protein fraction.

3. Method according to claim 2, comprising the further step of drying the washed plant tuber protein fraction.

4. Method according to claim 1, wherein the mechanical separation is carried out with a decanter.

5. Method according to claim 1, wherein the separation steps are carried out under oxidation-retardant conditions.

6. Tuber protein fraction with a medium molecular weight, producible according to claim 1, having a molecular weight between approximately 14 kD to 97 kD, with the bulk of the molecular weight distribution is between 20 kD to 60 kD.

7. Tuber protein fraction according to claim 6, wherein the tuber protein franction is a potato protein fraction with the following parameters:
pH: 4.0 to 6.0
Solubility: 2 to 8% in water with room temperature.

8. (canceled)

9. Method according to claim 1, wherein said precipitating of the medium-molecular-weight coagulated tuber plant protein fraction is performed by treating to a pH 2 to 7 and to a temperature at 80° C.

10. Method according to claim 5, wherein said oxidation-retardant conditions are obtained by one of adding reducing agents, working under protective gas and working in gas-tight plants.

11. Method according to claim 10, wherein said reducing agents are one of ascorbic acid and sodium bisulfite.
